(19) United States
(12) Patent Application Publication
(10) Pub. No.: US 2026/0144721 A1
(43) Pub. Date: May 28, 2026

(54) PREBIOTIC W/O MICROEMULSION FOR PRESERVATION OF PERSONAL CARE PRODUCTS

(71) Applicant: GALAXY SURFACTANTS LTD., Maharashtra (IN)

(72) Inventors: Nirmal KOSHTI, Piscataway, NJ (US); Bhagyesh Jagannath SAWANT, Maharashtra (IN); Pranali Rajendra JOSHI, Maharashtra (IN); Sohan PATIL, Maharashtra (IN); Bilal MOMIN, Maharashtra (IN); Parag Narendra SAVLA, Maharashtra (IN)

(21) Appl. No.: 19/398,741

(22) Filed: Nov. 24, 2025

(30) Foreign Application Priority Data
Nov. 28, 2024 (IN) 202421093155

(51) Int. Cl. A61K 8/06; A61K 8/34; A61K 8/368; A61K 8/37; A61K 8/44; A61Q 5/02; A61Q 19/00; A61Q 19/10

(52) U.S. Cl. CPC A61K 8/068; A61K 8/345; A61K 8/368; A61K 8/375; A61K 8/442; A61Q 19/00; A61K 2800/21; A61K 2800/524; A61Q 5/02; A61Q 19/10

(57) ABSTRACT

A water-in-oil microemulsion comprising water, glycerine, benzoic acid, glyceryl mono undecylenate (Formula I) and N-capryloyl amino acids (Formula II) is disclosed. The microemulsion is biodegradable, cold-processable, prebiotic (towards skin microbiota) preservative system for personal care compositions. The microemulsion of the present invention allows the incorporation of safe organic acids and their effective delivery for broad antimicrobial spectrum for preservation of personal care and cosmetic products with broad range of pH range. Further, a process for preparing the microemulsion and a method of preservation of personal care compositions are disclosed.

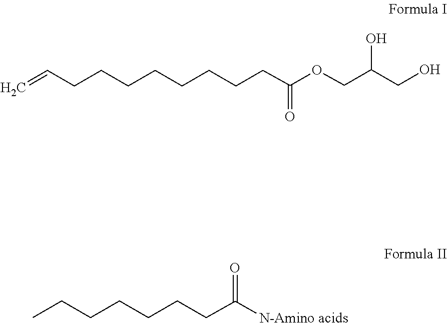

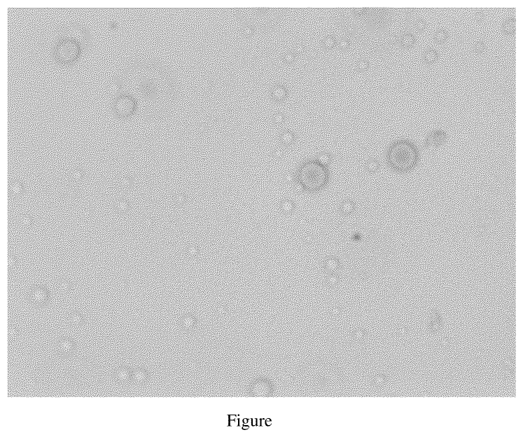

### CROSS REFERENCE TO RELATED APPLICATION

This application claims priority to Indian Patent Application No. 202421093155, filed on Nov. 28, 2024, the content of which is incorporated herein in its entirety.

### FIELD OF INVENTION

The present invention relates to a water-in-oil (W/O) microemulsion based preservative system for personal care compositions. More particularly, the present invention relates to water-in-oil type of microemulsion comprising of glyceryl mono undecylenate, N-capryloyl amino acid and benzoic acid as antimicrobial agents. The W/O microemulsion based preservative system is prebiotic towards skin microbiota. It is cold-processable, biodegradable and effective against wide range of microbes.

### BACKGROUND AND PRIOR ART OF THE INVENTION

Preservation of personal care products continues to be a challenge because the antimicrobials of yesteryears have been found to be dangerously toxic to both humans and ecology. Toxic effects of these diverse antimicrobials (for ex. halogenated molecules, isothiazolinones, parabens, formaldehyde releasers etc.) range from allergic reactions to neurotoxicity, endocrine disruption, reproductive toxicity and carcinogenicity. In view of this serious toxicity associated with most of the ‘effective’ antimicrobials of yesteryears, the personal care industry is trying to replace the toxic ones with the gentler antimicrobials (phenoxy ethanol, benzoic acid, sorbic acid, dehydroacetic acid, benzyl alcohol etc.; Cosmetics Directive Annex VI, https://echa.europa.eu/cosmetics-preservatives). Although these gentler antimicrobials listed as preservatives, they do have their limitations that are also reflected in the usage levels defined by ECHA. For example, benzyl alcohol has an intense odor and traces of benzaldehyde present, that lead to serious allergy. Phenoxy ethanol is generally considered not suitable for babies due to inadequate data on toxicology. (In September 2012, a risk assessment submitted by the French Agency ANSM, Agence nationale de sécurité des médicaments et des produits de santé) which raised concerns about the use of phenoxy ethanol as preservative in cosmetic products. ECHA limits use of phenoxy ethanol for baby care products to 0.4%. However, several manufactures of baby care products avoid phenoxy ethanol totally. For example, wet wipes for babies are preserved without using phenoxy ethanol (Seventh Generation, USA, P & G, The Honest Company, USA etc.). Preservation of wet wipes, particularly for babies, has been addressed with benzoic acid and keeping the pH around 4.0. Benzoic acid or sodium benzoate and dehydroacetic acid or sodium salt of dehydroacetic acid have their share of limitations. For leave-on applications, the allowed limit by ECHA for benzoic acid is 0.5%. Dehydroacetic acid chelates iron at ppm level and forms coloured complexes and hence it is avoided for colourless or white cream like formulations.

Microemulsion of antimicrobials are known to offer certain advantages of being easy to incorporate in home and personal care compositions for preservation of the same. The antimicrobials in microemulsified form are more efficacious due to their particle size and their ability to breach the cell membranes of microbes. Microemulsions easily fuse with the lipid bilayer of cell membrane and subsequently breach the cell membrane and enter the cytoplasm (‘A novel surfactant nanoemulsion with a unique non-irritant topical antimicrobial activity against bacteria, enveloped viruses and fungi’, James Baker et al. Microbiol. Res. 2001, 156, 1, University of Michigan), ‘Membrane Perturbation by Surfactant Candidates for STD Prevention’, Kyle Vanderelick, Langmuir, 2003, 19, 591 (Princeton University). U.S. Pat. No. 10,015,963 discloses water-in-oil type of microemulsions for preserving personal care products with water, phenoxy ethanol and organic acids. US '963 discloses use of N-acyl glycine's as emulsifiers for creating spontaneous formation of transparent microemulsion at pH of 6.5 or above. All microemulsions examples of US patent '963 (Table IV and V) are affected at pH 6.5 to 7.0. To achieve the formation of microemulsions disclosed in US patent '963, the antimicrobial organic acids are converted into their salt form and thereby significantly sacrificing their antimicrobial efficacy, particularly, in personal care formulations with pH of 6.0 to 7.0.

Commercially, this type of W/O microemulsion (U.S. Pat. No. 10,015,963) is available from Galaxy Surfactants Ltd., for example, Galguard Tetra which has water (˜25%), phenoxy ethanol (˜50%), benzoic acid, N-capryloyl glycine and N-undecylenoyl glycine and has pH of 6.5.

Thus, the major drawback of microemulsions with antimicrobial organic acids at near neutral pH, as described in the prior art, is the loss of antimicrobial activity. Organic acids with antimicrobial activity like N-acyl glycines, benzoic acid, or dehydroacetic acid etc. exist in their ionic carboxylate as the major form, leading to loss of acidic protons, and hence, very little carboxylic acid form is available and thereby their antimicrobial activity is seriously compromised.

For example, at pH 6.5 in aqueous medium, 90% benzoic acid is present in benzoate form. This is true with all ‘organic acid’ antimicrobials with carboxylic acid group. It should be noted that at neutral pH, the ‘organic acids type of antimicrobials’ lose their antimicrobial activity significantly. For spontaneous microemulsion formation, most of the N-capryloyl amino acids are used up as surfactant in their N-acyl carboxylate form (depending upon the base used) and rendering the microemulsion significantly less effective in terms of its antimicrobial activity. The effect of pH of such microemulsion on their antimicrobial activity is significant. The drop in the preservation performance of microemulsion, as disclosed in the prior art, with increase in the pH is drastic.

Thus, to avoid significant loss of antimicrobial activity of microemulsified ‘organic acids’ at neutral pH, the individual ingredients can be added in their native form separately to the personal care formulation. However, it is highly inconvenient to incorporate these water-insoluble organic acids into formulations even at warm temperatures. The organic acids like benzoic acid (m. p. 122° C.) or undecylenoyl glycine (m. p. 103° C.), capryloyl glycine (m. p. 109° C.) or dehydroacetic acid (m. p. 111° C.) are all water-insoluble solids with high melting points. Though finely divided powder form of these organic acids helps with uniform dispersion/distribution through the formulation, this ‘powder’ form is always associated with health hazard (inhalation) and the hazard of ‘dust-explosion’.

To address one or more above disclosed problems such as loss of antimicrobial efficacy and ease of operability, the present inventors have found a cold-processable microemulsion at acidic pH that provides uniform dispersion/distribution of the antimicrobial preservatives throughout the personal care formulation at room temperature. The inventors of the present invention have solved the problem by creating stable yet acidic W/O microemulsions of antimicrobials for their effective delivery and efficacy in the final personal care composition.

### Objectives of the Invention

It is an objective of the present invention to overcome the limitations of the prior art wherein the desired antimicrobial efficacy of safe organic acid type of antimicrobials is sacrificed when incorporated in at higher pH, however in present invention these are incorporated in the form of a microemulsion in low pH for effective delivery and preserve the personal care compositions.

It is another objective of the present invention to create an efficacious (synergistic), cold-processable, biodegradable preservation system based on ‘well-accepted’ personal care ingredients.

A further objective of the present invention is to create a preservative system for personal care compositions that would be prebiotic towards the human skin microbiota.

Another objective of the present invention is to create a broad-spectrum antimicrobial preservative system for personal care compositions that is effective at pH range of 4.0 to 7.0.

Yet another objective of the present invention is to create a preservation system for personal care compositions using principles of ‘Green Chemistry’ for the synthesis of component ingredients of the microemulsion.

Further objective of the present invention is to create a synergistic antimicrobial preservative system for personal care compositions that would avoid antimicrobial molecules with any functional group with likely potential to be cytotoxic like phenolic moiety (like phenoxy ethanol of prior art), halogens (chlorine, bromine, iodine), ‘difficult-to-biodegrade’ heterocycles and formaldehyde releasing functionalities etc.

### SUMMARY OF THE INVENTION

In an aspect the present invention is related to a water-in-oil microemulsion comprising:
a. 40% to 45% by weight of microemulsion, glyceryl mono undecylenate of Formula I;

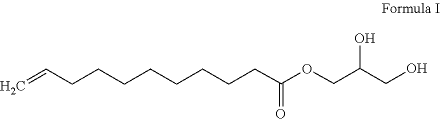 [SMILES: C=CCCCCCCCCC(=O)OCC(O)CO]

b. 16% to 22% by weight of microemulsion, an N-capryloyl amino acid of Formula II,

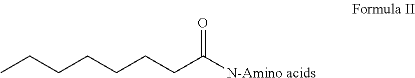

wherein amino acids are selected from glycine, L-proline, L-glutamic acid, L-glutamine, L-aspartic acid, L-asparagine, L-alanine, L-methionine, sarcosine;
c. 9% to 12% by weight of microemulsion, benzoic acid;
d. 16% to 25% by weight of microemulsion, glycerine; and
e. 10% to 15% by weight of microemulsion, water,
wherein the pH of the microemulsion is 4.0 to 4.5.

In another aspect the present invention also provides a process of preparing water-in-oil microemulsion, comprising glyceryl mono undecylenate; an N-capryloyl amino acid; benzoic acid; glycerine; and water, comprises the steps of:
a. suspending the N-capryloyl amino acid and benzoic acid to a stirred mixture of glyceryl mono undecylenate, glycerine and water to obtain a dispersion; and
b. adding a base to the stirred dispersion of step (a) to bring the pH to 4.0 to 4.5 to obtain a transparent water-in-oil microemulsion,
wherein the temperature of step (b) is maintained at 25° C. till the formation of water in oil microemulsion.

In yet another aspect the present invention relates to a personal care composition comprising:
a. a water-in-oil microemulsion, comprising glyceryl mono undecylenate; an N-capryloyl amino acid; benzoic acid; glycerine; water; and
b. one or more personal care ingredient,
wherein the component (a) is the only preservative system added in the composition.

In still another aspect the present invention relates to a water-in-oil microemulsion, for preservation of personal care formulations, comprising:
a. 40% to 45% by weight of microemulsion, glyceryl mono undecylenate of Formula I;

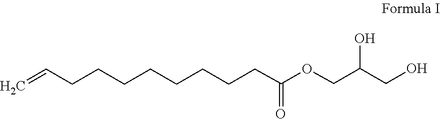 [SMILES: C=CCCCCCCCCC(=O)OCC(O)CO]

b. 16% to 22% by weight of microemulsion, an N-capryloyl amino acid of Formula II,

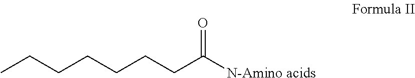

wherein amino acids are selected from glycine, L-proline, L-glutamic acid, L-glutamine, L-aspartic acid, L-asparagine, L-alanine, L-methionine, or sarcosine;
c. 9% to 12% by weight of microemulsion, benzoic acid;
d. 16% to 25% by weight of microemulsion, glycerine; and
e. 10% to 15% by weight of microemulsion, water,
wherein the pH of the microemulsion is between 4.0 to 4.5.

### BRIEF DESCRIPTION OF ACCOMPANYING FIGURE

FIGURE: Microscopic image of the water-in-oil microemulsion of the present invention showing the vesicles of size less than 1 micron with bilayers.

### DETAILED DESCRIPTION OF THE INVENTION

Antimicrobial organic acids exhibit moderate anti-microbial activity in their acid form, for example, benzoic acid, sorbic acid or dedydroacetic acid. Similarly, another class of antimicrobial organic acids are ‘N-acyl amino acids’ with moderate activity and biodegradability. Some of the N-acyl amino acids have been used to create antimicrobial combinations in the form of microemulsion for ease of operation and to enhance the effectiveness of constituent antimicrobials due to low micron size of particles. Liquid microemulsion are ‘easy-to-incorporate’ in the end personal care compositions and tiny particle size of microemulsion fuses easily with the lipids of bacterial cell membrane which ultimately results in overall breaching barrier of microbial cell. Since these salts of N-acyl amino acids antimicrobials are used as emulsifiers for creating the microemulsions, in the process the microemulsion results in significant loss of antimicrobial activity of ‘organic acid type’ of antimicrobials.

This major drawback is addressed by the compositions of the present invention wherein it is demonstrated that transparent ‘water-in-oil’ microemulsions at acidic pH can be made with organic acids type antimicrobials and are more efficacious compared to the ‘water-in-oil’ microemulsions of the prior art (U.S. Pat. No. 10,015,963). This has been achieved by creating microemulsion with glyceryl mono undecylenate, N-octanoyl amino acid or N-capryloyl amino acid, benzoic acid, glycerine and water in low pH, without use of phenolic moiety like phenoxy ethanol of prior art, which have potential toxicity.

The terms “water-in-oil microemulsion”, “W/O microemulsion” or microemulsion which have the same meaning as known in the art, are used synonymously in the specifications of the present invention and have same meaning as to composition in the water-in-oil microemulsion form, which is completely transparent (isotropic) and formed spontaneously (thermodynamically stable) at room temperature.

### Glyceryl Mono Undecylenate:

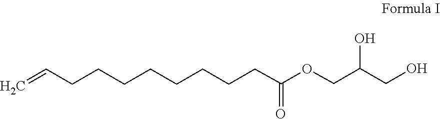 [SMILES: C=CCCCCCCCCC(=O)OCC(O)CO]

Glyceryl mono undecylenate (Formula I, CAS No 65684-27-7) forms the major component, component ‘a’, of the microemulsions of the present invention. Glyceryl mono undecylenate is known to personal care industry and is used as an emulsifier and as an emollient in personal care formulations. It is known for its anti-yeast property. It is synthesized from glycerine and undecylenic acid, both raw materials originate from renewable sources. The esterification between glycerine and undecylenic acid can be affected by conventional chemical catalysis or biocatalysis. Commercially, it is available as mixture of mono esters (two regio-isomers), diesters (two regio-isomers) and triester. Commercial glyceryl mono undecylenate has mono ester content of about 50%. However, changing the stoichiometry of reactants and catalyst, it is possible to get higher mono ester content up to 80% or even above. It is a pale yellow liquid at room temperature and gets converted into soft solid at temperatures lower than 15° C. It is a water insoluble material with water solubility of 8.5-182.5 μg/L at 20° C. (pH=6.3) (ECHA). Synthesis of glyceryl mono undecylenate with mono content of 74% is described in Example 1 with base catalysis whereas Example 2 describes synthesis of glyceryl mono undecylenate with 60% mono content using solid supported, immobilized lipase enzyme and successful recycling of the enzyme catalyst.

In an embodiment the glyceryl mono undecylenate of Formula I is present in an amount equivalent to about 30% to 60% by weight of the microemulsion. Preferably, glyceryl mono undecylenate of Formula I is present in an amount of 40% to 50% by weight of the microemulsion. Preferably glyceryl mono undecylenate of formula I is present in an amount of up to 40% or up to 41% or up to 42% or up to 43% or up to 44% or up to 45% by weight of the total water-in-oil microemulsion of the present invention. Most preferably the amount of glyceryl mono undecylenate of Formula I is present in any amount of 40% to 45% by weight of water-in-oil microemulsion of the present invention.

### N-Capryloyl Amino Acids:

The second important component, component ‘b’, of the microemulsion of the present invention is N-capryloyl amino acid. These organic acids, at least some of them, are known for their antimicrobial activity and are exploited by personal care industry. Of the several N-capryloyl amino acids, the most widely used is N-capryloyl glycine (CAS No. 14246-53-8). Its action against some of microbes of skin microbiota (Table 3 of Example 3) that exacerbate the skin condition when it erupts due to other factors. For example, Acne vulgaris and skin resident Cutibacterium acnes or seborrheic dermatitis/dandruff and the resident yeast species of Malassezia. It has moderate antimicrobial action against yeast of Candida genera and against Gram positive bacteria of Staphylococci and Corynebacteria genera of axillary vault that degrade odourless sweat into malodorous molecules. It is used by personal care industry for anti-acne, anti-dandruff, anti-malodour and feminine intimate hygiene products. This lipidated glycine resembles fatty acids of skin and its acidic nature offers derma-protection for the restoration of the skin's acid mantle. (Human skin's acidic nature prevents colonization of skin by pathogenic microbes). Thus, N-capryloyl glycine is well established as skin benefit agents and the leaders of personal care industry (L'Oreal, Estee Lauder, J & J and Unilever etc.) are using it in their offerings. Commercially, N-capryloyl glycine is available as powder and has melting point of around 106° C. Green synthesis of N-capryloyl glycine and its minimum inhibitory concentration against a variety of microbes is given in the Table 3 of Example 3. Here again, wet powder of N-capryloyl glycine can be used in the making of water-in-oil microemulsion without further drying as explained in Example 3.

Other N-capryloyl amino acids that are reported in literature are N-capryloyl sarcosine (Cas No 2421-32-1, U.S. Pat. No. 11,304,886), N-capryloyl L-glutamic acid (Cas No 31462-7-4), N-capryloyl L-glutamine (CAS No 65684-27-7, IN202421009600), N-capryloyl L-proline (IN202421064802). It is interesting to note that some are solids like N-capryloyl L-glutamine, N-capryloyl glycine and N-capryloyl L-glutamic acid whereas N-capryloyl sarcosine, N-capryloyl L-proline and are liquids at room temperature. The literature reveals that these N-capryloyl amino acids exhibit gentle antimicrobial activity (Table 1 below). Also, physical and spectral data (IR, proton and carbon ¹³NMRs) of some other hitherto unknown N-capryloyl amino acids (N-capryloyl L-alanine, N-capryloyl L-valine, N-capryloyl L-isoleucine) have been given in Example 5.

In an embodiment the N-capryloyl amino acids, component ‘b’, of water-in-oil microemulsion of the present invention is present in the range of 10% to 25% by weight of microemulsion. More preferably the component ‘b’ is present in an amount of up to 16% or up to 17% or up to 18% or up to 19% or up to 20% or up to 21% or up to 22% by weight of the microemulsion. Most preferably the component ‘b’ is present in the range of between 16% to 22%.

In an embodiment the N-capryloyl amino acids, component ‘b’, of water-in-oil microemulsion of the present invention are selected from one or more N-capryloyl glycine, N-capryloyl sarcosine, N-capryloyl L-glutamic acid, N-capryloyl L-glutamine, N-capryloyl L-proline, N-capryloyl L-alanine, N-capryloyl L-valine, and N-capryloyl L-isoleucine and mixture thereof.

TABLE 1
Minimum inhibitory concentration of N-capryloyl amino acids.

| | N- | N- | N- | N- |
|---|---|---|---|---|
| | Capryloyl | Capryloyl | Capryloyl | Capryloyl |
| | glycine | L-glutamine | L-proline | sarcosine |
| Staphylococcus | 1.0 | 0.3 | 0.3 | 0.7 |
| aureus | | | | |
| ATCC 6538 | | | | |
| Cutibacterium acnes | 1.0 | 0.7 | 0.4 | 0.6 |
| ATCC 6919 | | | | |
| Escherichia coli | 0.7 | 0.3 | 0.5 | 0.7 |
| ATCC 8739 | | | | |
| Pseudomonas | 1.0 | 0.3 | 0.5 | 1.0 |
| aeruginosa | | | | |
| ATCC 15442 | | | | |
| Candida albicans | 0.6 | 0.2 | 0.4 | 0.7 |
| ATCC 10231 | | | | |
| Malassezia furfur | 0.4 | 0.3 | 0.3 | 0.6 |
| ATCC 14521 | | | | |
| Aspergillus niger | 1.0 | 0.4 | 1.0 | >1.0 |
| ATCC 16404 | | | | |

It is found that glyceryl mono undecylenate, N-capryloyl L-glutamine, N-capryloyl L-proline and N-cocoyl L-proline show selectivity in antimicrobial action, showing bactericidal effect against pathogenic Staphylococcus aureus while being gentle on Staphylococcus epidermidis which is a major commensal bacterium involved in the first line of defence with skin (host) cells.

### Benzoic Acid:

Benzoic acid, component ‘c’, (CAS No 65-85-0) is one of the oldest and relatively safe antimicrobial preservatives. It is used in preservation of food products (fruit juices, sauces and ketchups) and preservation of baby wet wipes where the pH of wet wipes is around 4.0. It is biodegradable and free from any adverse toxicology effect. Allowed concentration of benzoic acid in personal care products for the purpose of preservation is 0.5%.

In an embodiment the component ‘c’, benzoic acid, of water-in-oil microemulsion of present invention, is present in the range of 5% to 15% by weight of the microemulsion. Preferably, the component ‘c’ is present in an amount of up to 9%, or up to 10%, or up to 11%, or up to 12% by weight of the microemulsion of the present invention. In most preferable embodiment the benzoic acid is present in the range of 9% to 12% by total weight of water-in-oil microemulsion of the present invention.

To get the maximum preservation efficacy of the water-in-oil microemulsion of the present invention, the benzoic acid is present in the form of native acidic form.

### Glycerine:

Glycerine forms component ‘d’ of the water-in-oil microemulsion of the present invention. It is very popular humectant, moisturizer (CAS No 56-81-5) used by personal care industry. It is a bio renewable ingredient derived from triglycerides of fatty acids.

In an embodiment the component ‘d’ of the water-in-oil microemulsion of the present invention is present in the range of 10 to 20% by weight of the water-in-oil microemulsion. Preferably the component d is present in an amount of up to 10% or up to 11% or up to 12% or up to 13% or up to 14% or up to 15% or up to 16% or up to 17% or up to 18% by weight of the water-in-oil microemulsion of the present invention. Most preferably the component d is present in the range of 12 to 18% by weight of the water-in-oil microemulsion of the present invention.

### Water:

Water is the component ‘e’ of the water-in-oil microemulsion of the present invention. In an embodiment the water is present in the range of 8% to 12% by weight of the water-in-oil microemulsion. Preferably the water-in-oil microemulsion of the present invention comprises water in an amount of up to 8% or up to 9% or up to 10% or up to 11% or up to 12% by total weight of microemulsion.

### Preparation of Water-In-Oil Microemulsions:

Microemulsions of the present invention are spontaneously prepared as per the procedure described in Example 4. It is formed by simply mixing all the constituents together at room temperature and the pH is adjusted to 4.0 to 4.3 by alkali (inorganic bases). Typical microemulsion has glyceryl mono undecylenate about 40% by weight, about 20% N-capryloyl amino acid, about 10% benzoic acid, about 20% glycerine and about 10% water. The pH is adjusted to 4.0 to 4.3 to get the spontaneous transparent water-in-oil free flowing (viscosity is <1000 cps) microemulsion with active matter content of around >70% by weight of the total microemulsion. The microemulsion of Example 4 uses N-capryloyl glycine along with glyceryl mono undecylenate, benzoic acid, glycerine and water. The acidic microemulsion is stable at room temperature and several cycles of freezing and thawing does not induce or trigger any instability of breaking of microstructure. Preferably, the microemulsion of the present invention is frozen to 0° C. for 24 hours and followed by thawing the frozen microemulsion to 25° C. for 24 hours. After the multiple freeze and thaw cycles the microemulsion of present invention was found to withstand these extreme temperature conditions and was stable.

Instead of one N-capryloyl amino acid, one can use more than one N-capryloyl amino acids or even N-acyl amino acids to create microemulsion using the general procedure given in Example 4 and 5. It is known that the N-acyl amino acids comprises several fatty acyl derivatives of amino acids where fatty acyl is derived from fatty acids having carbon chain length of 6 to 24 carbons. Several N-capryloyl amino acids have been used to prepare the microemulsion of the present invention. Examples include N-caprylolated naturally occurring amino acids like L-glutamic acid, L-alanine, L-glutamine and L-proline (Table 5 of Example 5), N-capryloylated non-chiral amino acid like glycine and non-natural amino acid like sarcosine, are exploited to create isotropic microemulsions of the present invention. It is observed that when the bulk of substituent group on the chiral carbon or on the nitrogen increases to induce more hydrophobicity (non-polarity) then the transparent and stable microemulsions are not feasible. Thus, N-capryloyl L-valine or N-capryloyl L-isoleucine do not yield the stable microemulsion.

In an embodiment, a process of preparing water-in-oil microemulsion, comprising glyceryl mono undecylenate; an N-capryloyl amino acid; benzoic acid; glycerine; and water, is disclosed. In an embodiment the process to prepare microemulsion according to the present invention comprises steps:
a. suspending the N-capryloyl amino acid and benzoic acid to a stirred mixture of glyceryl mono undecylenate, glycerine and water to obtain a dispersion; and
b. adding a base to the stirred dispersion of step (a) to bring the pH between 4.0 to 4.5 to obtain a transparent water in oil microemulsion,
wherein the temperature of step (b) is maintained between 20° C. to 30° C. till the formation of water-in-oil microemulsion.

In an embodiment the N-capryloyl amino acid is selected from one or more N-capryloyl glycine, N-capryloyl sarcosine, N-capryloyl L-glutamic acid, N-capryloyl L-glutamine, N-capryloyl L-proline, N-capryloyl L-alanine, N-capryloyl L-valine, and N-capryloyl L-isoleucine, N-cocoyl L-proline and mixture thereof. Preferably the suspending step is carried out in a reaction vessel equipped with overhead stirrer that are well known in art.

Preferably the base being added in step b is selected from a group comprising of strong and weak bases, so as to raise the pH to desired level, preferably between 4.0 to 4.5.

Preferably bases used to adjust the pH can be organic or inorganic bases. Most preferably the inorganic bases are incorporated to adjust the pH of dispersion of step a.

Preferable inorganic bases are selected from but not limited to sodium hydroxide, potassium hydroxide, or ammonium hydroxide.

Preferably the pH adjustment in step b is carried out at any temperature between 20° C. to 30° C. Most preferably the pH adjustment in step b is carried out at a temperature of around 25° C.

In an embodiment the particle size of the water-in-oil microemulsion of the present invention is analyzed by Differential Light Scattering and polarized optical microscopy. The microemulsions of the present invention are bicontinuous (in their concentrated form, 70% active) and upon dilution in water it gives vesicles.

The microemulsion of Example 4, upon 100 times dilution with water, results in vesicles with water inside the core of bilayer wall.

The size of these vesicles ranges from 500 nm to 1000 nm according to differential light scattering analysis. The zeta potential of the vesicles is found in the range from −40 to −43 Mv, indicating that these aggregates are stable (would not coalesce very easily to form bigger aggregates). This is also confirmed by microscopy (1000× magnification) with staining using methylene blue. The double layered vesicle with aqueous core is seen clearly (FIGURE). These less than a micron-sized antimicrobial vesicles with ‘bilayered’ wall fuse with the ‘bilayered’ cell membrane (bilayer of amphiphilic molecules) of microbes and ultimately breach it.

### Significance of Acidic pH of Microemulsion:

As discussed in the background section, the prior art teaches us the utility of antimicrobials like N-acyl amino acids for creating microemulsion wherein alkaline earth metal salts of N-acyl amino acids behave like surfactants. Spontaneous, stable and energy efficient microemulsions are formed with desired particle size (U.S. Pat. No. 10,015,963).

However, the serious disadvantages of these microemulsions of antimicrobials is that at pH 6.5 and above, the N-acyl amino acids lose their antimicrobial efficacy significantly.

The present composition, however, provides for the micro emulsion at pH 4-4.5 where the preservation efficacy is demonstrated even after elevating pH to neutral and near neutral levels of 5.5 to 6.5 in the end personal care formulation.

Preservation of Personal Care Products with Microemulsion of the Present Invention:

In an embodiment, the water-in-oil microemulsion for preservation of personal care formulations is disclosed. The microemulsion of the present invention can be incorporated in the personal care formulation in the range of about 0.5% to about 2.5% by weight of the personal care formulation. Preferably the water-in-oil microemulsion is incorporated in an amount of up to 2.0% or up to 1.8% or up to 1.6% or up to 1.5% or up to 1.4% or up to 1.2% or up to 1.0% or up to 0.8% or up to 0.6% or up to 0.5% of the total weight of the personal care formulation. In most preferable embodiment, the water-in-oil microemulsion of the present invention is incorporated at any concentration between 1.0% to 2.0% by weight of the personal care formulations of the present invention.

In an embodiment the personal care formulation can be a group of formulations allowed to be left on human body, that is ‘leave-on’ formulations or can be a group of formulations that are meant to be rinsed off from human body by water washing, that is ‘rinse-off’ formulations. In some embodiments the water-in-oil microemulsion of the present invention can by incorporated in various cosmeceuticals, dermatological and pharmaceutical formulation for topical application.

In an embodiment the personal care formulations are selected from the group, but not limited to hand wash, body wash, face wash, shampoo, conditioner, shampoo in solid formats, conditioner in solid formats, soap bar, syndet bar, emulsions, creams, deodorant, anti-perspirant, gels, paste, serum, wet wipes, baby wipes, baby care formulations, lotions, sun care formulations, oral care formulations, formulations for intimate hygiene, a range of hair care formulations, and a range of body care formulations.

To those skilled in art, a range of personal care formulations can be preserved with microemulsions of the present invention and the present disclosure of the personal care formulations shall not limit the scope of present invention.

In an embodiment, preservation efficacy of water-in-oil microemulsions of the present invention is evaluated by using microemulsion of present invention in a shampoo, body wash, cream and on wet wipes. For preservation efficacy test of personal care formulations as well as for wet wipes, are performed as per the protocols prescribed by PCPC (Personal Care Product Council, Washington, USA). All four products namely shampoo, body wash, cream and on wet wipes pass the preservation efficacy test.

In yet another embodiment, the water-in-oil microemulsion of the present invention give the ease of formulation by allowing the formulator to incorporate the water-in-oil microemulsion of the present invention without requirement of high processing temperature. Preferably the water-in-oil microemulsion of the present invention are incorporated into the personal care formulations at room temperature. This not only provides an ease of incorporation to formulator but also contributes towards the conversation of energy by avoiding the high temperature processes for preparing the topical formulations.

### Prebiotic Property of the Microemulsion Containing Glyceryl Mono Undecylenate, N-Acyl Amino Acids and Benzoic Acid Towards Skin-Microbiota:

In an embodiment, the microemulsion of the present invention provides a prebiotic property towards the skin microbiota.

Staphylococcus epidermidis and Staphylococcus aureus are part of skin microbiota. In natural environment Staphylococcus epidermidis is a symbiont and prevents colonization of highly infectious Staphylococcus aureus which is contagious and causes serious diseases. It spreads by direct contact with an infected person or by using a contaminated object or by inhaling infected droplets dispersed by sneezing or coughing. Skin infections from Staphylococcus aureus are common, and the bacteria can spread through the blood stream to infect distant organs. Skin infections may cause blisters, abscesses (furuncles), redness and swelling (Cellulitis) in the infected area. Staphylococcus aureus infects hair roots (Folliculitis) by developing a small pimple at the base of the hair. It also causes fluid filled blisters (Impetigo) on skin that burst. Staphylococcus aureus also causes Mastitis of breast after 1 to 4 weeks of the delivery, which may include cellulitis and abscesses. The area around the nipple becomes red and painful. Abscesses often release large numbers of bacteria into the mother's milk. The bacteria may then infect the nursing infant. In children, Staphylococcus aureus causes epidermal necrolysis that results in peeling of skin. Toxins of Staphylococcus aureus also result in food poisoning and ‘toxic shock syndrome’. Some species are also known to release coagulase enzyme that results in blood clotting. Once it enters blood then it results in osteomyelitis or pneumonia. Also, many strains, that originate from hospital or from hospital staff, are antibiotic resistant and hence any antimicrobial that has decent activity against Staphylococcus aureus is welcome especially for topical application where resident Staphylococcus aureus can be the cause for not only skin/hair infections but other systemic infections and subsequent human to human spreading of the infections.

Thus, Staphylococcus aureus of skin microbiota is pathogenic and in contrast to this, the Staphylococcus epidermidis is commensal and involved in many host benefitting actions.

Staphylococcus epidermidis help maintain the skin barrier by producing ceramides, which are a key component of the epithelial barrier. It does this by secreting a sphingomyelinase, which helps the skin produce ceramides and provides nutrients for the bacteria. This process helps prevent skin dehydration and aging.

Staphylococcus epidermidis prevents colonization of Staphylococcus aureus by secreting serine protease that destroys the biofilm of Staphylococcus aureus (Iwase et al.; Nature, Letters Vol 465, p 246-250 (2010)). ‘Modulins’ produced by Staphylococcus epidermidis, along with host produced AMPs, selectively inhibit pathogens like Staphylococcus aureus and Streptococci. Staphylococcus epidermidis is also involved in immunity pathway signalling and in anti-inflammatory activity by secreting lipoteichoic acid. (E. A. Grice and J. A. Segre, Nature Reviews: Microbiology, Volume 9, 243-253 (2011)).

It is also important to note that the biofilm forming property of infectious Staphylococcus aureus is supported by Propionibacterium acnes that produces coproporphyrin III to promote aggregation of Staphylococcus aureus (Julia Segre et al. Nature Reviews, vol 116, p 143-155, (2018)). The antimicrobials combination of the present invention shows a good activity against Cutibacterium acnes (Table 4 of Example 4), and this indirectly helps prevent formation of biofilms by Staphylococcus aureus. Thus, the prebiotic combination of glyceryl mono undecylenate, N-capryloyl amino acid and benzoic acid of the present invention generates a prebiotic situation on the skin by creating higher population of commensal Staphylococcus epidermidis which in turn suppress pathogenic Staphylococcus aureus by way of generating antimicrobial peptides as well as by generating film destroying enzyme and by biosynthesizing antibiotics. Thus, the W/O microemulsion of glyceryl mono undecylenate, N-capryloyl amino acid and benzoic acid of the present invention has huge potential to create a prebiotic effect on skin via personal care formulations, particularly via ‘leave-on’ applications (cream formulation of Example 10).

Microemulsions of the present invention wherein glyceryl mono undecylenate is combined with N-capryloyl L-glutamine or N-capryloyl L-proline or N-cocoyl L-proline, exhibits good prebiotic properties (IN202421064802). Highly selective antimicrobial (prebiotic) activity of N-cocoyl L-proline was first reported by Galaxy Surfactants Ltd. (the applicant) during a seminar talk (17 Apr. 2024, In-cosmetics Global, Paris) on Galseer DermaGreen, a prebiotic oil-soluble cleanser for dry and sensitive skin.

Thus, the synergistic combination of antimicrobial preservatives of the present invention is a bio-degradable and prebiotic system towards skin with other additional benefits derived from it like anti-dandruff, anti-acne and anti-malodor activity.

### Advantages of the Invention

### 1) Prebiotic Preservation System for Personal Care

The microemulsions are prebiotic towards skin microbiota. They display remarkable selectivity in the antimicrobial property while dealing with pathogenic and skin-friendly symbiont bacteria.

### 2) Broad Spectrum of Antimicrobial Activity at Wide Range of pH

The microemulsion of the present invention is effective against bacteria, yeast and mold. It is also effective in the pH range 4.0 to 7.0. Acidic microemulsion of the present invention exhibits the maximum synergy amongst the component members.

### 3) Microemulsion as Effective Delivery Form of Antimicrobials for Preservation

Antimicrobials in microemulsion form, easily fuse with the lipid bilayers of microbial cell membrane, thereby breaching it to allow easy passage of antimicrobials into cytoplasm. This enhances the antimicrobial efficacy of solid antimicrobials that are often both oil and water insoluble. Simple dilution of W/O microemulsion results in vesicle-liposome like structure with two layered lipid walls. This can be easily observed under polarized optical microscope with 1000× magnification power. The liposome like structures can be stained with methylene blue to see the aqueous blue core surrounded by lipid bilayers. These kind of liposomes with lipid bilayers fuse with the lipid bilayer of microbes and enhancing their antimicrobial efficacy.

### 4) Personal Care Benefits in Addition to Preservation

N-capryloyl amino acids and glyceryl mono undecylenate are both very effective against acne aggravating Cutibacterium acnes, seborrheic dermatitis aggravating Malassezia species, sweat degrading (mal-odor causing) Staphylococcus hominis and Corynebacteria. (Minimum inhibitory concentrations against a variety of microbes relevant to personal care (Table 4 in Example 4).

### 5) Cold-Processibility: Ease of Incorporation

The microemulsion of the present invention is a very thin liquid and easy to incorporate in all sorts of personal care formulations including leave-on, wash-off compositions and wet-wipes.

### 6) Transparent Formulations

Transparent personal care formulations (shampoo, face wash, body wash etc.) are possible at the use level of 1.0 to 2.0% for preservation purpose. Incorporation of antimicrobials via microemulsion form makes it feasible to create transparent formulations even though the antimicrobials (glyceryl mono undecylenate, benzoic acid and N-capryloyl amino acid) are water-insoluble in their native form.

7) Free from Known Toxic Antimicrobials (Free from any Potential Toxic Functionality in the Constituent Members)

It is free from toxic antimicrobials like parabens, formaldehyde releasers or halogenated molecules or heterocycles.

### 8) Biodegradable

All the components (N-capryloyl amino acids, glyceryl mono undecylenate, benzoic acid, glycerine) of the microemulsion are biodegradable.

### 9) Energy Efficient Formation of Microemulsion

W/O microemulsions are affected instantaneously by simply mixing all the ingredients together and adjusting the pH to 4.0 to 4.3. Very short period of agitation (mixing) at room temperature (cold processibility) makes preparation of microemulsion very energy efficient. Furthermore, the microemulsion of the present invention is also easy to incorporate in range of personal care formulations.

10) Globally Compliant with the Regulatory Bodies

All the components that are used in preparation of microemulsion are globally used personal care ingredients, namely, glycerine, glyceryl mono undecylenate, and known N-capryloyl amino acids like N-capryloyl glycine and benzoic acid.

### EXAMPLES

The present invention is now described by way of non-limiting illustrative examples. The details of the invention provided in the following examples are given by way of illustration only and should not be construed to limit the scope of the present invention.

All ingredients and surfactants (except PEG 150 distearate, GLDA, Cetostearyl alcohol and glyceryl monostearate) are procured from Galaxy Surfactants Ltd, Mumbai, India and are without any antimicrobial preservatives. Sodium cocoyl isethionate is solid surfactant without any antimicrobial preservative. Other anionic surfactants (sodium cocoyl glutamate, sodium cocoyl taurate etc.) are aqueous solutions and used without any antimicrobial formulations. Amphoteric cocamidopropyl betaine is without any antimicrobial preservative. Non-ionic lauryl glucoside is without any preservative. Polyethylene glycol 150 distearate in flake form is used as rheology modifier. Stearoyl lactylate is used as moisturizer. 1,3 propane diol distearate is a pearlizer. Methoxy cinnamidopropyl behenyl dimethyl ammonium chloride is quaternary UV-absorber cum hair conditioner. GLDA (Tetra Sodium Glutamate Diacetate) is a green chelating agent.

The lipase enzyme is procured from Advanced Enzymes, India

The MICs (minimum inhibitory concentrations) of N-acyl L-prolines and N-acyl L-glutamines and their combinations against various microbes are determined as per PCPC (Personal Care Product Council, Washington, USA) protocol.

### Example 1: Synthesis of Glyceryl Mono Undecylenate

A mixture of undecylenic acid (400 g, 2.17 mole), potassium hydroxide (4.2 g, 0.25% w/w of batch size, dissolved in 200 g of water) and Glycerine (1000 g, 10.85 mole) are heated at 160° C. with good stirring under steady flow of nitrogen. The progress of reaction is monitored from the amount of water collected as well as the drop in the acid value of the reaction mass. At the end of the reaction, the temperature is lowered to 100° C. and transferred to a 2 L separatory flask. Upon standing, the glycerol layer (615 g), separated from the ester product as pale-yellow liquid (720 g), contained 32% glycerol. Glyceryl monoundecylenate with 32% glycerine is used in the preparation of the microemulsion of Example 4. A small sample was (5.0 g) further washed with water to remove glycerine (up to levels<1.0%) and then analyzed for sap value, acid value and gas chromatography and to determine minimum inhibitory concentration.

Acid value: 3.5 and Sap value: 213.

FTIR: 1738 (carbonyl), 2851, 2925 (C—H stretch) and 3381 broad (OH) cm⁻¹.

Gas Chromatography: Agilent Gas chromatography (model number 7890B) coupled with FID. Capillary Column: HP-5MS UI (30 m×0.25 mm×0.25 micron), Injector temp: 250° C., Detector temp: 300° C., carrier gas: Helium 1 cc/min, column temperature programming: 30° C. to 300° C. with 15° C./min.

Gas chromatographic analysis reveals the glyceryl mono undecylenate content to be 74%, glyceryl diundecylenate 22% and glyceryl triundecylenate 3% and free undecylenic acid<2%.

Below are minimum inhibitory concentrations against the microbes, covering bacteria, yeast and mold.

TABLE 2
Minimum inhibitory concentrations of glyceryl mono
undecylenate against bacteria, yeast and mold.

| Microorganisms | MIC in % |
|---|---|
| Gram positive | |
| Staphylococcus aureus ATCC 6538 | 0.1 |
| Cutibacterium acnes ATCC 6919 | 0.9 |
| Staphylococcus epidermidis NCIM 2493 | 1.0 |
| Staphylococcus hominis MTCC 8980 | 0.7 |
| Micrococcus luteus NCIM 2103 | 0.5 |
| Corynebacterium xerosis ATCC 373 | 0.5 |
| Gram variable | |
| Gardnerella vaginalis ATCC 14018 | 0.3 |
| Gram negative | |
| Escherichia coli ATCC 8739 | 1 |
| Pseudomonas aeruginosa ATCC 15442 | 1 |
| Burkholderia cepacia ATCC 25416 | 1 |
| Yeast | |
| Candida albicans ATCC 10231 | 1 |
| Candida glabrata ATCC 66032 | 0.4 |
| Malassezia furfur ATCC 14521 | 0.2 |
| Malassezia restricta ATCC MYA 4611 | 0.2 |
| Mold | |
| Aspergillus niger ATCC 16404 | 0.8 |

Interesting observation to be noted from this Table 2 is, glyceryl mono undecylenate has very good activity against Gram positive pathogenic Staphylococcus aureus (MIC, 0.1%). It shows practically useful activity against yeast Candida glabrata (MIC, 0.4%), Gardnerella vaginalis (MIC, 0.3%) and Malassezia species (MICs, 0.2%).

### Example 2: Synthesis of Glyceryl Mono Undecylenate Using Lipase Enzyme

A mixture of undecylenic acid (100 g, 0.54 gmol), glycerine (150 g, 1.62 gmol) and immobilized lipase (Candida antartica) catalyst (5 g, Addzyme 015, 10 Advanced Enzymes, India) is stirred under nitrogen and reduced pressure of 20 mmHg at 65-70° C. The progress of the reaction is monitored by the drop in the acid value of the reaction mixture. The reaction mixture is cooled down to room temperature after the acid value of less than 2 is achieved (9 h) and the catalyst is removed by filtration. The filtered product is allowed to phase-separate, and the lower level of glycerine is removed. Recovered glycerine and the isolated catalyst is recycled for the next batch and thus, seven additional experiments are performed by recycling the catalyst.

Sap value: 230, Acid value: 1.0, glycerine content: 3.0%, Iodine value=100. Color: 35 APHA.

FTIR: 1737 cm⁻¹ (carbonyl), 2857 cm⁻¹, 2928 cm⁻¹ (C—H stretch) and 3405 (OH) cm⁻¹.

Mono ester of this glyceryl mono undecylenate is found to be 60%.

The catalyst is recycled seven times successfully with similar results of batch cycle time and the % of mono undecylenate content in the product was found to be in rage of around 60%.

It can be seen from Example 1 and 2 that at the end of the reaction excess glycerine separates and settles down. The excess glycerine used in the synthesis does not separate quantitatively. About 30% glycerine remains in the product and it can be used for making microemulsion without any further purification.

### Example 3: Synthesis of N-Capryloyl Glycine

To a stirred mixture of glycine (76.5 g, 1.02 gmol) in water (235.5 g) at 25° C., sodium hydroxide solution (168.5 g of 48. % aqueous solution, 2.02 gmol) and capryloyl chloride (165.8 g, 1.0 gmol) are added simultaneously while maintaining temperature between 15 to 25° C. and pH between 11.0 to 12.0. The addition takes 2 to 3 hours depending on the efficiency of temperature control. The reaction mass is stirred for an additional period of two hours. It is further acidified by addition of concentrated hydrochloric acid. The precipitated solid is filtered and washed with water (525 ml) to remove the mineral acidity. The lipidated glycine is obtained as wet solid powder (215 g) with 10% water. The wet coarse powder is used in the preparation of microemulsions of the present invention (Example 4). A small sample (3 g) of this wet cake of capryloyl glycine is dried after vacuum drying at 65° C. The dried powder with, <1.0 moisture, has acid value of 277.5 and M.P. of 105-107° C. The HPLC analysis of dried powder indicated 98% purity as N-capryloyl glycine with 2.0% caprylic acid.

### Minimum Inhibitory Concentration Numbers of N-Capryloyl Glycine (at pH 5.0) Against a Variety of Microbes Relevant to Personal Care Applications.

TABLE 3
Minimum Inhibitory concentration of N-capryloyl glycine (at pH
5.0) against microbes relevant to personal care applications.

| | MIC (in %) |
|---|---|
| Gram positive | |
| Staphylococcus hominis MTCC 8980 | 0.3 |
| Staphylococcus epidermidis NCIM 2493 | 1 |
| Staphylococcus aureus ATCC 6538 | 1 |
| Micrococcus luteus NCIM 2103 | 1 |
| Corynebacterium xerosis ATCC 373 | 1 |
| Propionibacterium acnes MTCC 1951 | 1 |
| Gram negative | |
| Escherichia coli ATCC 8739 | 0.7 |
| Pseudomonas aeruginosa ATCC 15442 | 1 |
| Burkholderia cepacia ATCC 25416 | 1 |
| Yeast | |
| Candida albicans ATCC 10231 | 0.6 |
| Candida glabrata ATCC 66032 | 1 |
| Malassezia furfur ATCC 14521 | 0.4 |
| Malassezia restricta ATCC-MYA 4611 | 0.4 |
| Mold | |
| Aspergillus niger ATCC 16404 | 1 |

### Example 4: Preparation of W/O Microemulsion at pH 4.2 with Glyceryl Mono Undecylenate, Capryloyl Glycine, Benzoic Acid, Glycerine and Water

N-Capryloyl glycine (wet powder with ˜10% water, 20 g from Example 3), benzoic acid in powder form (10 g), are suspended in stirred glyceryl mono undecylenate (60 g, containing 32% glycerine, from Example 1) and water (7 g) at room temperature. To this stirred mixture, sodium hydroxide lye (3 g, 48%) is added dropwise till the pH of the mixture reaches up to 4.0 to 4.2 resulting into formation of pale yellow homogeneous and transparent microemulsion.

Analysis microemulsion: Water content 10.0%, pH as is 4.2, glycerine content 19.0%, viscosity: 200 cps, measured at 25° C.

The microemulsion of Example 4, upon dilution (100 times) with water, results in the formation of vesicles with water inside the core of bilayered wall.

The size of these vesicles ranges from 700 nm to 1000 nm according to differential light scattering analysis. The zeta potential of the vesicles ranges from −40 to −43 Mv.

TABLE 4
Minimum inhibitory concentration of individual components
and w/o microemulsion of present invention.

| | Glyceryl | N- | | Micro- |
|---|---|---|---|---|
| | mono | capryloyl | Benzoic | emulsion |
| | undecylenate | Glycine | acid | Example 4 |
| Microorganisms | MIC in % | MIC in % | MIC in % | MIC in % |
| Staphylococcus | 0.1 | 1 | 0.8 | 0.1 |
| aureus | | | | |
| ATCC 6538 | | | | |
| Cutibacterium | 0.9 | 1 | 0.8 | 0.1 |
| acnes ATCC 6919 | | | | |
| Escherichia coli | 1 | 0.7 | 1.0 | 0.3 |
| ATCC 8739 | | | | |
| Pseudomonas | 1 | 1 | 0.6 | 0.5 |
| aeruginosa | | | | |
| ATCC 15442 | | | | |
| Burkholderia | 1 | 1 | 0.9 | 0.2 |
| cepacia | | | | |
| ATCC 25416 | | | | |
| Candida albicans | 1 | 1 | 0.8 | 0.2 |
| ATCC 10231 | | | | |
| Malassezia furfur | 0.2 | 0.4 | 0.5 | 0.1 |
| ATCC 14521 | | | | |
| Aspergillus niger | 0.8 | 1 | 1.0 | 0.7 |
| ATCC 16404 | | | | |

The Table 4 above shows the minimum inhibitory concentration against the typical microbes that include bacteria, yeast and mold. Surprisingly, the unexpected synergy is observed in case of microemulsion.

It is evident that the minimum inhibitory concentration for microemulsion of individual antimicrobial exhibits moderate activity. However, when combined together in the form of a microemulsion the MIC numbers are enhanced compared to individual antimicrobial. The lowering of MIC numbers against Cutibacterium acnes, Malassezia yeast and against Candida yeast suggests the synergism of different components of water in oil microemulsion of the present invention.

Equally surprising is the synergy exhibited by the microemulsion against Gram negative bacteria (Table 4, MICs against, Escherichia coli, Burkholderia cepacia and Pseudomonas aeruginosa). The unexpected synergy is very prominent for Gram negative Escherichia coli against which the individual antimicrobial exhibits minimum inhibitory concentration ranging from 0.7 to 1.0% whereas the microemulsion of Example 4 exhibits MIC of 0.3%.

Similar observation is also seen in the case of yeast Candida albicans wherein microemulsion inhibits the growth of yeast at 0.2%.

The microemulsion of this Example 4, upon 100 times dilution with water, results in vesicles with water inside the core of bilayer wall.

### Example 5: General Procedure for Synthesis of N-Capryloyl Amino Acid

N-Capryloyl amino acids are synthesized by reacting capryloyl chloride with an amino acid in aqueous medium in the presence of stochiometric quantity of inorganic base (Schotten Baumann reaction). Amino acids reacted are L-glutamic acid, L-glutamine, L-aspartic acid, L-asparagine, L-proline, L-alanine, L-valine, L-isoleucine and sarcosine.

To a stirred mixture of respective α-amino acid (1.02 gmol) in water (sufficient to create final product with 40 to 45% concentration) at 25° C. under nitrogen, sodium hydroxide solution (48. % aqueous solution, 2.02 gmol) and capryloyl chloride (1.0 gmol) are added simultaneously while maintaining temperature between 15 to 25° C. and pH between 11.0 to 12.0. The addition takes 2 to 3 hours depending on the efficiency of temperature control (removal of exotherm). The reaction mass is stirred for an additional period of two hours. It is further acidified by addition of concentrated hydrochloric acid or sulphuric acid. The separated N-acyl amino acid is phase-separated if it is liquid or it is filtered if it is solid. It is washed with water, regardless of its physical form, to remove the acidity of mineral acids. The washed products are further dried under vacuum.

In case of N-capryloyl L-glutamic acid, the reaction mass does not phase-separate after acidification and hence it is extracted with ethyl acetate. Removal of ethyl acetate yields N-capryloyl L-glutamic acid as off-white solid. Only two N-capryloyl amino acids are liquid in nature, namely, N-capryloyl sarcosine and N-capryloyl L-proline.

N-capryloyl L-alanine, N-capryloyl L-valine, N-capryloyl L-isoleucine are solids and are precipitated in from of their aqueous solution of sodium salt after the N-acylation step. The solids are washed with water and dried. The physical data and spectral data are given in the tables below.

TABLE 5
Physical properties of corresponding N-acyl amino acids

| N-Capryloyl amino acid | Physical properties |
|---|---|
| N-Capryloyl glycine | White solid, 105-107° C. |
| N-Capryloyl L-glutamic acid | Pale yellow solid, 73-75° C. |
| N -Capryloyl L-proline | Pale yellow liquid with |
| | solidification point <05° C. |
| N-Capryloyl sarcosine | Pale yellow liquid with |
| | solidification point <05° C. |
| N-Capryloyl L-glutamine | white solid, mp 120-124° C. |
| N-Capryloyl L-alanine | White powder, mp 80-82° C. |
| N-Capryloyl L-valine | White solid, mp 125-126° C. |
| N Capryloyl L-isoleucine | White solid, 110-112° C. |

Physical and spectral data of hitherto unknown N-capryloyl L-amino acids.

N-Capryloyl L-alanine: m.p., 80-82° C., FTIR (neat): 1640 cm⁻¹, 1733 cm⁻¹, 1702 cm⁻¹, 2923 cm⁻¹, 3355 cm⁻¹.

¹H NMR (500 MHz), δ (in ppm): 0.83 (3H, t, J=7.0 Hz), 1.21 to 1.24 (8H, m), 1.38 (3H, t, J=7.5 Hz), 1.56 (2H, t, J=8 Hz), 2.19 (2H, t, J=8 Hz), 4.46 (1H, t, J=7 Hz), 6.82 (1H, d, J=6.5 Hz).

¹³C NMR (500 MHz), δ (in ppm): 14, 17.95, 22.55, 25.65, 28.95, 29.05, 29, 05, 29.14, 31.63, 316.29, 48.51, 174.34, 176.39.

N-Capryloyl L-valine: m.p. 125-126° C., FTIR (neat): 1608 cm⁻¹, 1698 cm⁻¹, 2930 cm⁻¹, 3327 cm⁻¹.

¹H NMR (500 MHz), δ (in ppm) 0.85 (3H, t, J=6.5 Hz), 0.89-0.95 (8H, m), 1.24 to 1.28 (8H, m), 1.60-1.62 (2H, m), 2.18-2.32 (3H m), 4.58 (1H, q, J=3.5 Hz), 6.33 (1H, d, J=8 Hz).

¹³C NMR (500 MHz), δ (in ppm): 11.57, 14.01, 15.30, 22.55, 24.55, 24.04, 25.77, 28.93, 29.12, 31.64, 34.05, 36.53, 37.73, 56.43, 174.32, 175.21.

N-Capryloyl L-isoleucine: m.p. 110-112° C., FTIR (neat): 1620 cm⁻¹, 1694 cm⁻¹, 2928 cm⁻¹, 2857 cm⁻¹, 3334 cm⁻¹.

¹H NMR (500 MHz), δ (in ppm): 0.83 to 0.96 (7H, m), 1.13 to 1.43 (9H, m), 1.46 to 1.94 (1H+2H, m) 1.90 to 1.94 (1H, m), 2.22 to 2.31 (2H, m), 4.60 (1H, d, J=5 Hz), 6.42 (1H, d, J=8.5 Hz), 10.86 (1H, s).

¹³C NMR (500 MHz), δ (in ppm): 14.03, 17.64, 18.93, 0.56, 25.78, 28.93, 29.00, 29.13, 31.06, 31.06, 31.64, 36.60, 57.00, 174.44, 175.14.

### Example 6: Microemulsions with N-Capryloyl Amino Acids with Glyceryl Mono Undecylenate, Benzoic Acid, Water and Glycerine as Per the Disclosure of the Present Invention

TABLE 6a
Microemulsions with N-capryloyl amino acids with glyceryl
mono undecylenate (GMU), benzoic acid, glycerine and water
(Composition as per the present invention 6A to 6F).

| | Microemulsion according to the present invention | | | | | |
|---|---|---|---|---|---|---|
| | (Example 6A to 6F) | | | | | |
| | A | B | C | D | E | F |
| Ingredients | % | % | % | % | % | % |
| GMU (70% mono) | 40 | | | | 40 | 40 |
| GMU (60% mono) | | 40 | 40 | | | |
| GMU (50% mono) | | | | 40 | | |
| N-Capryloyl Glycine | 18 | | 18 | 18 | | |
| N-Capryloyl Sarcosine | | | | | 18 | |
| N-Capryloyl L-Proline | | | | | | 18 |
| N-Cocoyl L-Proline | | | | | | |
| N-Capryloyl L-Glutamic acid | | | | | | |
| N-Capryloyl L-Glutamine | | | | | | |
| N-Capryloyl L-alanine | | 18 | | | | |
| Benzoic acid | 10 | 10 | 10 | 10 | 10 | 10 |
| Glycerine | 20 | 20 | 20 | 20 | 20 | 20 |
| Water | 9 | 9 | 9 | 9 | 9 | 9 |
| NaOH 48% | 3 | 3 | 3 | 3 | 3 | 3 |
| pH as such | 4.2 | 4.2 | 4.2 | | 4.2 | 4.2 |
| Nature | Clear | Clear | Clear | Clear | Clear | Clear |

TABLE 6b
Microemulsions with N-capryloyl amino acids with glyceryl mono undecylenate
(GMU), benzoic acid, glycerine and water (Composition as per the
present invention 6G to 6J) and Comparative Example 6K.

| | Microemulsion according to the present invention | | | | Comparative |
|---|---|---|---|---|---|
| | (Example 6G to 6J) | | | | Example 6K |
| | G | H | I | J | K |
| Ingredients | % | % | % | % | % |
| GMU (70 mono) | | 40 | 40 | 45 | 40 |
| GMU (60% mono) | 40 | | | | |
| GMU (50% mono) | | | | | |
| N-Capryloyl Glycine | | | | | 18 |
| N-Capryloyl Sarcosine | | | | | |
| N-Capryloyl L-Proline | | | | | |
| N-Cocoyl L-Proline | 18 | | | | |
| N-Capryloyl L-Glutamic acid | | 18 | | | |
| N-Capryloyl L-Glutamine | | | 18 | 18 | |
| N-Capryloyl L-alanine | | | | | |
| Benzoic acid | 10 | 10 | 10 | 10 | 10 |
| Glycerine | 20 | 20 | 20 | 20 | 10 |
| Water | 9 | 9 | 9 | 12 | 3 |
| NaOH 48% | 3 | 3 | 6 | 3 | 12 |
| pH as such | 4.30 | 4.25 | 4.30 | 4.25 | 6.50 |
| Nature | Clear | Clear | Clear | Clear | Clear*** |

***slow phase separation is seen after 6 hours at room temperature.
It is found that N-Capryloyl L-valine and N-capryloyl L-isoleucine when used in similar compositions as above, do not yield transparent microemulsions at room temperature spontaneously.

However, spontaneous microemulsions are affected with several other N-capryloyl amino acids as depicted in Examples 6A to 6K. The microemulsion of Example 6A and comparative example 6K is made with glyceryl mono undecylenate with mono ester content of 70% and N-capryloyl glycine; whereas 6C and 6D are made with glyceryl mono undecylenate with mono ester content of 60% and 50% respectively.

Other examples of Table 6a and 6b deploy N-capryloyl amino acids made from naturally occurring amino acids, namely, N-capryloyl L-alanine (6B), N-capryloyl L-proline (6F), N-capryloyl L-glutamic acid (6H), N-capryloyl L-glutamine (6I). Non-natural amino acid derivative, N-capryloyl sarcosine (6E). N-Cocoyl L-proline is also shown to give instantaneous microemulsion (6G).

N-cocoyl L-proline is reported to be selective between pathogenic Staphylococcus aureus and host-benefitting Staphylococcus epidermidis (IN202421064802). Supporting host-defending commensal Staphylococcus epidermidis whose main job is to prevent colonization by pathogenic Staphylococcus aureus is tantamount to indirect prebiotic effect of molecules like N-cocoyl L-proline and glyceryl mono undecylenate which is also reported to be favourably selective in its action to be a prebiotic towards skin microbiota (U.S. Ser. No. 18/817,316).

### Example 7: Antimicrobial Efficacy of Microemulsion of pH 4.2 (Example 6A) and Same Microemulsion with Adjusted pH of 6.5 (Example 6K)

N-Capryloyl glycine (dry powder, 18 g), benzoic acid in powder form (10 g), are suspended in stirred glyceryl mono undecylenate (40 g), and water (3 g) at room temperature. To this stirred mixture, sodium hydroxide lye (12 g, 48% aqueous solution) is added dropwise till the pH of the mixture reaches 6.5 (Example 6K) resulting into formation of pale yellow homogeneous and transparent microemulsion. It remained transparent and homogeneous for a very short time. Analysis: Water content 10.0%, pH 6.5, glycerine content 19.0%

Preservation Efficacy of Body Wash with Microemulsions of Example 6A and Example 6K

TABLE 7A
‘Sulphate-free’ bodywash formula.

| Ingredients | Weight % |
|---|---|
| Phase A | |
| Water | To make 100.0 |
| Sodium cocoyl glutamate (30%) | 7.00 |
| Sodium cocoyl isethionate (85%, powder) | 3.00 |
| Lauryl polyglucoside (50%) | 8.00 |
| Cocoamidopropyl betaine (36%) | 18.00 |
| Tetrasodium glutamate diacetate | 0.30 |
| Phase B | |
| Glyceryl mono laurate | 2.0 |
| Polyethylene glycol 150 distearate (100% solid) | 1.0 |
| 3- Propane diol distearate (100% solid) | 1.5 |
| Phase C | |
| Microemulsion of Example 4/Example 6A | 1.5 |
| or Example 6K (70% active) | |

The separate bodywashes are preserved with 1% active of microemulsions of Example 6A and Example 6K respectively and pH of both bodywash compositions is adjusted to 5.5.

TABLE 7B
Preservation efficacy test for bodywash composition
preserved with microemulsion of 6A.

| | Initial | | | |
|---|---|---|---|---|
| | inoculum | 0 day | 7 days | 14 days |
| Staphylococcus | 1.3 × 10⁸ | 1.6 × 10⁶ | 1 × 10³ | <10 |
| aureus | | | | |
| ATCC 6538 | | | | |
| Cutibacterium | 1.1 × 10⁸ | 3.5 × 10⁶ | 3 × 10¹ | <10 |
| acnes ATCC 6919 | | | | |
| Escherichia coli | 2.6 × 10⁸ | 5.2 × 10⁶ | <10 | <10 |
| ATCC 8739 | | | | |
| Pseudomonas | 1.2 × 10⁸ | 1.3 × 10⁶ | <10 | <10 |
| aeruginosa | | | | |
| ATCC 15442 | | | | |
| Burkholderia | 2.2 × 10⁸ | 2.9 × 10⁶ | 7 × 10¹ | <10 |
| cepacia | | | | |
| ATCC 25416 | | | | |
| Candida albicans | 2.4 × 10⁶ | 3.2 × 10⁵ | 5.3 × 10² | <10 |
| ATCC 10231 | | | | |
| Malassezia furfur | 2.5 × 10⁶ | 6.2 × 10⁴ | <10 | <10 |
| ATCC 14521 | | | | |
| Aspergillus niger | 2.1 × 10⁶ | 6.8 × 10⁴ | 4.7 × 10² | 1.1 × 10² |
| ATCC 16404 | | | | |

TABLE 7C
Preservation efficacy test for Bodywash composition
preserved with microemulsion of 6K.

| | Initial | | | |
|---|---|---|---|---|
| | inoculum | 0 day | 7 days | 14 days |
| Staphylococcus | 1.3 × 10⁸ | 4.5 × 10⁶ | 7.7 × 10³ | 4.4 × 10² |
| aureus | | | | |
| ATCC 6538 | | | | |
| Cutibacterium | 1.1 × 10⁸ | 3.9 × 10⁶ | <10 | <10 |
| acnes ATCC 6919 | | | | |
| Escherichia coli | 2.6 × 10⁸ | 3.8 × 10⁶ | 1 × 10⁴ | 3.9 × 10³ |
| ATCC 8739 | | | | |
| Pseudomonas | 1.2 × 10⁸ | 6.2 × 10⁶ | 2 × 10¹ | <10 |
| aeruginosa | | | | |
| ATCC 15442 | | | | |
| Burkholderia | 2.2 × 10⁸ | 4.4 × 10⁶ | 9.3 × 10³ | 1.5 × 10³ |
| cepacia | | | | |
| ATCC 25416 | | | | |
| Candida albicans | 2.4 × 10⁶ | 6.7 × 10⁶ | 1.4 × 10³ | <10 |
| ATCC 10231 | | | | |
| Malassezia furfur | 2.5 × 10⁶ | 1.7 × 10⁶ | <10 | <10 |
| ATCC 14521 | | | | |
| Aspergillus niger | 2.1 × 10⁶ | 1.6 × 10⁴ | 2.9 × 10³ | 7.8 × 10³ |
| ATCC 16404 | | | | |

### Significance of Acidic pH of Microemulsion:

To demonstrate the better efficacy over those of U.S. Pat. No. 10,015,963, where microemulsion of desired particle size having alkaline earth metal salts of N-acyl amino acids acting surfactants are taught but show declining antimicrobial efficacy at pH 6.5 and above, the present microemulsions were tested at different pH.

For the purpose two identical microemulsions with the exact same formula but differing the overall pH of the microemulsion were tested. In one case pH is adjusted to 4.2 (Example 6A) and in case of other pH is adjusted to 6.5 in line with U.S. Pat. No. 10,015,963 (Example 6K). These two microemulsions with two different pHs are then used at 1% active level (microemulsions are 70% active) to check the preservation efficacy of microemulsion in a ‘sulphate-free’ body wash as per Table 7A, with pH adjusted 5.5 after addition of microemulsions of 6A and 6K.

Preservation efficacy at pH 5.5 is evaluated on 7^{th} day and on 14^{th} day. Body wash formulation with acidic microemulsion (Example 6A) passes the 7^{th} day criteria of preservation efficacy test (3 log reduction for bacteria and 1 log reduction for yeast and mold, Table 7B) whereas the body wash composition preserved with near neutral microemulsion (Example 6K) does not meet the criteria for 7^{th} day. It fails to meet 7^{th} day criteria, and it continues to fail on 14^{th} day too (Table 7C).

Accordingly, it is found that in most preferred embodiments the pH of the microemulsion is between 4.0 to 4.5, which when used in personal care formulations maintains the preservation efficacy even at higher pH.

Further the viscosity of the microemulsion of the present invention is below 1000 cps when measured at room temperature. The viscosity of the microemulsion of example 4 is around 200 cps, measured at 25° C. The viscosity of other microemulsion of example 6B to 6J is in between 200-400 cps. The viscosity of the microemulsion of the present invention is such that it is easy to flow and incorporate it into personal care formulations or any formulations in general.

### Example 8: Preparation of Transparent Shampoo and its Preservation with Microemulsion of Example 4

TABLE 8
Transparent shampoo formulation and its preservation
with microemulsion of Example 4.

| Ingredients | % |
|---|---|
| Water | To make 100 |
| Sodium cocoyl isethionate ( powder, 85% active) | 2.0 |
| Sodium cocoyl glutamate (30%) | 10% |
| Sodium cocoyl taurate (40%) | 15.0 |
| Cocomidopropyl betaine (36%) | 13.0 |
| Glycerine (100%, liquid) | 2.0 |
| PEG 150 DS (100% solid) | 1.5 |
| Sodium stearoyl lactylate (100% solid) | 2.0 |
| Methoxy cinnamidopropyl behenyl dimethyl | 0.5 |
| ammonium chloride (needles, solid) | |
| Microemulsion of Example 4 (70% active, liquid) | 2.0 |
| Tetrasodium glutamate diacetate (GLDA) (50% solution) | 0.5 |
| Adjusted pH | 6.0 |
| Viscosity | 11500 cps |

Procedure: A mixture of all water soluble surfactants and water are mixed together and warmed to 70° C. To it sodium cocoyl isethionate, sodium stearoyl lactylate, methoxy cinnamidopropyl behenyl dimethyl ammonium chloride and polyethylene glycol 150 distearate are added and stirring is continued until a homogeneous mass is obtained. It is then cooled to 25° C. and to it other ingredients like glycerine, GLDA and microemulsion of Example 4 as preservative are added and stirring is continued till homogeneous mass is obtained. The pH is adjusted down to 6.0 using citric acid. The resultant mixture has viscosity of 11500 cps at 25° C. The composition clears the preservation efficacy test as per the PCPC guidelines.

### Example 9: Preparation of Bodywash and its Preservation with Microemulsion of Example 4

TABLE 9
Bodywash formulation and its preservation
with microemulsion of Example 4.

| Ingredients | Weight % |
|---|---|
| Phase A | |
| Water | To make 100.0 |
| Sodium cocoyl glutamate (30%) | 7.00 |
| Sodium cocoyl isethionate ( 85%, solid) | 3.00 |
| Lauryl polyglucoside (50%) | 8.00 |
| Cocamidopropyl betaine (36%) | 18.00 |
| Tetrasodium glutamate diacetate | 0.30 |
| Phase B | |
| Glyceryl mono laurate (solid, 100%) | 2.0 |
| Polyethylene glycol 150 distearate | 1.5 |
| Micro emulsion of Example 4 (70% active) | 2.0 |
| 1, 3- Propane diol distearate | 2.00 |
| Adjusted pH with citric acid | 5.51 |
| Viscosity | 8670 cps |

Procedure: Phase A is prepared separately by gentle stirring of the components at 60° C. The components of phase B are added one by one to stirred mass of phase A and agitation is continued at 60° C. till the mass becomes homogeneous. It is cooled to ambient temperature and pH of the stirred mass is adjusted to 5.5 with citric acid solution. After ensuring the uniformity of pH, colour and fragrance are added and stirring continued till the mass becomes homogeneous. The resultant formulation has viscosity of 8670 cps at 25° C. Using same formula another batch is made, and the pH is adjusted to 7.0. Both compositions (one at pH 5.5 and pH 7.0) pass the preservation efficacy test.

### Example 10: Preparation of Cream and its Preservation with Microemulsion of Example 4

TABLE 10
Cream formulation and its preservation
with microemulsion of Example 4.

| Ingredients | Weight % |
|---|---|
| Phase A | |
| Water | To make 100.0 |
| Glycerine | 4.0 |
| Polyethyleneglycol-7-glyceryl cocoate | 2.0 |
| Sodium gluconate | 0.2 |
| Tetrasodium glutamate diacetate | 0.2 |
| Phase B | |
| Cetostearyl alcohol | 7.0 |
| Stearic acid | 2.0 |
| Glyceryl monostearate | 5.0 |
| Isopropyl myristate | 2.5 |
| Phase C | |
| Microemulsion of Example 4 (70% active) | 2.0 |

Procedure: Water-miscible ingredients (Phase A) are mixed together and heated to 75° C. under stirring. Similarly oil soluble ingredient are mixed together and stirred separately at 75° C. Phase B is then added to Phase A with constant stirring and homogenized (Silverson) for 30 minutes until emulsion is obtained. The cream is then cooled to room temperature and Phase C (microemulsion of Example 4) is added and stirring is continued until uniform consistency is obtained. pH of the final formulation is adjusted to 6.0 and optional fragrance and color are blended uniformly. The resultant formulation has viscosity of 5000 cps at 25° C. The composition passes the preservation efficacy test.

### Example 11: Preservation Efficacy of Microemulsion of Example 4 in Wet Wipes for Babies

Preparation of 2.1% dispersion for wet wipes: Considering the original microemulsion is 70% active, 12 ml of original microemulsion was diluted with 388 mL of sterile distilled water, pH was adjusted to 4.3, total volume of solution was made to 400 mL.

### Preparation of Wet Wipes:

Non-woven cut wipes are (34 wipes each weighing 1.28 g) treated with 2.1% active solution/dispersion of microemulsion of Example 4 (2.1% active, pH 4.3) to yield wetted and loaded weight of each wipe around 4.8 g. This means 4.8-1.28=3.52 g of total increase in weight due to payload of diluted microemulsion. This amounts to loading of 1.5% active matter of antimicrobial considering the original microemulsion is 70% active. The loaded wet wipes are kept for 60 mins and then used for preservation efficacy test (The Challenge Test). The pH of wet wipes is 4.3 after loading the antimicrobial solution (or dispersion).

The preservation efficacy test is performed against Staphylococcus aureus, Escherichia coli, Pseudomonas aeruginosa, Burkholderia cepacia, Candida albicans, and Aspergillus brasiliensis. Wipes, each weighing approximately 4 g, is placed in sterilized 250 ml glass bottles under aseptic conditions. It is inoculated, by adding 40 μl (equivalent to 1% of culture based on total weight of a wipe) of 24-hour-old cultures (bacteria 10⁸, yeast and mold 10⁶ CFU/ml), to each 4 g wipe. On the 0-day, TAT (Tryptone Azolectin Tween 20) broth is added to the bottles at a 1:10 dilution (4 g wipe+40 μl culture+36 ml TAT broth), and the bottles is vortexed. Samples are taken for plating and dilution, considering 10¹ to 10⁶ dilutions. From each dilution, 1 ml is plated onto Modified Letheen agar for bacteria and Sabouraud Dextrose agar for yeast/mold. Results are recorded after 48 and 96 hours, and the number of colonies is counted to calculate the reduction in the number of microorganisms. Same procedure is repeated on 7^{th}, 14^{th}, 21^{st}, and 28^{th} days.

Wet wipes with active loading of 1.5% of antimicrobials using microemulsion of Example 4 clears the preservation efficacy test.

The preservation efficacy of water-in-oil microemulsions of the present invention is evaluated by using microemulsion of Example 4 in a shampoo, body wash, cream and on wet wipes. For preservation efficacy test of personal care formulations as well as for wet wipes, are performed as per the protocols prescribed by PCPC (Personal Care Product Council, Washington, USA).

‘Sulphate-free’ shampoo with 6.0 pH is preserved with 1.4% of microemulsion of Example 4. The preparation of shampoo formulation is described in Example 8. Bodywash with pH of 5.5 is prepared as per the procedure given in Example 9. Example 10 gives formula for a cream which is preserved with 1.4% of microemulsion of Example 4. In all the formulations the microemulsion is added when the formulation mass is close to room temperature. It is thoroughly mixed till homogeneous and finally pH is adjusted to desired pH. In case of wet wipes, the preservation is affected (Example 11) by loading 1.4% concentration of the total ‘mix’ and pH of this mix is adjusted to 4.5.

The preservation efficacy is evaluated as per PCPC M5 protocol. All four examples (Example 8 to Example 11) pass the preservation efficacy test.

### Example 12: Prebiotic Property of Microemulsion of Example 4 and Example 6G

A combination of glyceryl mono undecylenate, N-capryloyl glycine and benzoic acid in the form of a microemulsion (Example 4 or Example 6A) is evaluated for establishing highly selective action against pathogenic and highly infectious Staphylococcus aureus (causes several skin infections, see ‘detailed discussion’) in the presence of Staphylococcus epidermidis. A prebiotic substance supports the good microbes (beneficial to host skin cells) of human microbiota that are also actively involved in what is called ‘natural’ or ‘innate’ immunity in protecting humans from pathogenic microbes. The microemulsion of Example 4 does support commensal, host-benefiting bacterium Staphylococcus epidermidis by selectively going aggressively against the pathogenic Staphylococcus aureus which competes with Staphylococcus epidermidis for both space and food. Staphylococcus epidermidis secretes certain enzyme that destroys the biofilm created by Staphylococcus aureus. It also produces ‘modulins’ that kill pathogenic Staphylococcus aureus. S. epidermidis is also involved in signalling mechanism connected to immune response of skin.

In-vitro evaluation of prebiotic property of the microemulsion of Example 4 and Example 6G) using Staphylococcus epidermidis NCIM 2493 as the commensal bacterium and Staphylococcus aureus ATCC 6538 as the pathogenic bacterium of skin microbiota.

### Sample Preparation:

Dispersion of microemulsion of Example 4 (1.0% in distilled water) is prepared with a pH of dispersion around 5.5 and used immediately. Dilute solution of sod hydroxide can be used to adjust the pH of dispersion to 5.5.

The test strain suspensions (Staphylococcus aureus and Staphylococcus epidermidis) are each adjusted individually to a bacteria count of approximately 10⁴ CFU/ml.

The test sample (500 μl or mg, 1% dispersion of Example 4 is mixed (vortex mixer) with PBS (5.2 ml) and TSB (300 μl) broth. Subsequently, the above prepared microbial suspensions of Staphylococcus aureus (100 μl) and Staphylococcus epidermidis (500 μl) are inoculated to the test sample with PBS and TSB. The ‘control’ tube containing PBS (500 μl) in place of the test substance is included. Incubation is carried out with orbital shaker (120 rpm) for 4 hrs (for ‘leave-on’ products like creams or lotions) and 15 mins (‘rinse-off’ products like body wash/face wash) at 37° C.

The incubated sample (500 μl) is removed and centrifuged (13,000 rpm) and the pellet is redispersed in PBS (500 μl).

The redispersed material (500 μl) is added to 4.5 ml of TSB for serial dilution and CFU/ml is determined by plating on Tryptic Soy Agar. The colonies of Staphylococcus epidermidis appear as creamish-white that can be easily distinguished from golden yellow colonies of Staphylococcus aureus on Tryptic Soy Agar plates.

In control, Staphylococcus aureus count is 3.9×10⁶ and Staphylococcus epidermidis count is 2.5×10⁷ (see the Table 11A) However, in the ‘test’ set, the count for Staphylococcus aureus is reduced to 1.9×10⁵ whereas the count for Staphylococcus epidermidis is observed to be 1.9×10⁷ when compared to what is observed in case of the ‘control’. Thus, in the presence of microemulsion of Example 4, the count of pathogenic Staphylococcus. aureus is reduced within four hours of contact time as compared to Staphylococcus epidermidis. In terms of bacterial growth inhibition in terms of ‘log reduction’, it is observed to be 1.32 for Staphylococcus aureus and 0.10 for Staphylococcus epidermidis. Similar bacterioselective action is seen with microemulsion of Example 6G wherein N-capryloyl glycine is replaced with N-cocoyl L-proline (Table 11B).

TABLE 11 A
Antimicrobial selectivity of microemulsion of Example 4.

| | S. aureus | S. epidermidis |
|---|---|---|
| Control (cfu/ml) | 3.9 × 10⁶ | 2.5 × 10⁷ |
| Test (cfu/ml) | 1.9 × 10⁵ | 1.9 × 10⁷ |
| Log reduction | 1.32 | 0.10 |

TABLE 11 B
Antimicrobial selectivity of microemulsion of Example 6G.

| | S. aureus | S. epidermidis |
|---|---|---|
| Control (cfu/ml) | 4.9 × 10⁵ | 1.2 × 10⁷ |
| Test (cfu/ml) | 3.8 × 10⁴ | 5.2 × 10⁶ |
| Log reduction | 1.10 | 0.36 |

Two W/O microemulsions (Example 4/Example 6A and Example 6G) are evaluated, through in-vitro test, for selective antimicrobial action against pathogenic Staphylococcus aureus and host-friendly Staphylococcus epidermidis in the presence of each other.

In this experiment, the contact time with the mixed culture of both organisms is 4 h. It can be seen from the Tables 11A and 11B that after 4 h of contact time the count for Staphylococcus aureus is reduced by more than 1.0 log in case of both examples (Example 4 and 6G) whereas the count of commensal Staphylococcus epidermidis is very little affected (0.1 to 0.3 log reduction, Tables 11A and 11B in Example 12).

We claim:

1. A water-in-oil microemulsion comprising:
a. 40% to 45% by weight of microemulsion, glyceryl mono undecylenate of Formula I; 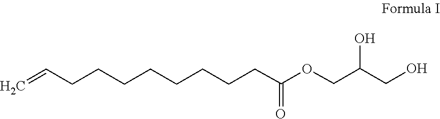 [SMILES: C=CCCCCCCCCC(=O)OCC(O)CO]
b. 16% to 22% by weight of microemulsion, an N-capryloyl amino acid of Formula II, 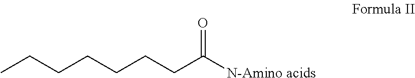
wherein amino acids are selected from glycine, L-proline, L-glutamic acid, L-glutamine, L-aspartic acid, L-asparagine, L-alanine, L-methionine, sarcosine;
c. 9% to 12% by weight of microemulsion, benzoic acid;
d. 16% to 25% by weight of microemulsion, glycerine; and
e. 10% to 15% by weight of microemulsion, water,
wherein the pH of the microemulsion is 4.0 to 4.5.

2. The water-in-oil microemulsion as claimed in claim 1, wherein the weight percentage of components (a), (b) and (c) together is at least 65% by total weight percentage of the microemulsion.

3. The water-in-oil microemulsion as claimed in claim 1, wherein the microemulsion is transparent liquid having viscosity of less than 1000 cps when measured at 25° C.

4. The water-in-oil microemulsion as claimed in claim 1, wherein the microemulsion is stable when subjected to multiple cycles of freezing at 0° C. for 24 h and thawing at 25° C. for 24 h.

5. A process of preparing water-in-oil microemulsion, comprising glyceryl mono undecylenate; an N-capryloyl amino acid; benzoic acid; glycerine; and water, comprises the steps of:
a. suspending 16% to 22% by weight of N-capryloyl amino acid and 9% to 12% by weight of benzoic acid to a stirred mixture of glyceryl mono undecylenate of 40% to 45% by weight, glycerine 16% to 25% by weight and water 10% to 15% by weight to obtain a dispersion; and
b. adding a base to the stirred dispersion of step (a) to bring the pH between 4.0 to 4.5 to obtain a transparent water-in-oil microemulsion,
wherein the temperature of step (b) is maintained at 25° C. till the formation of water-in-oil microemulsion.

6. A personal care composition comprising:
a. a water-in-oil microemulsion, comprising glyceryl mono undecylenate; an N-capryloyl amino acid; benzoic acid; glycerine; water of claim 1; and
b. one or more personal care ingredient,
wherein the component (a) is the only preservative system added in the composition, and wherein the weight % of the water-in-oil microemulsion is from 1% to 2.5% by weight of the total personal care composition.

7. The personal care composition as claimed in claim 6, wherein the personal care composition is shampoo, body wash, face wash, hair conditioner, hair serum, soap bar, syndet bar, cream, lotion, hygiene wash, deodorant, anti-perspirant, a cosmeceutical and wet wipe.
